(19) United States
(12) Patent Application Publication
(10) Pub. No.: US 2006/0223098 A1
(43) Pub. Date: Oct. 5, 2006

(54) Circularizable nucleic acid probes and amplification methods

(76) Inventors: David J. Lane, Rockport, MA (US); James Smith, Westford, MA (US); Thomas Beals, Acton, MA (US)

Correspondence Address: STROOCK & STROOCK & LAVAN LLP, 180 MAIDEN LANE, NEW YORK, NY, 10038, US

(21) Appl. No.: 11/393,553

(22) Filed: Mar. 30, 2006

Related U.S. Application Data
(60) Provisional application No. 60/667,781, filed on Mar. 31, 2005.

(51) Int. Cl. C12Q 1/68; C12P 19/34

(52) U.S. Cl. 435/6; 435/91.2

(57) ABSTRACT

The present invention relates to methods for preparing linear circularizable nucleic acid probes and/or circular nucleic acid probes. Such probes may be used as the starting material for rolling circle amplification (RCA) and/or ramification-extension amplification (RAM) of nucleic acid molecules. The present invention further provides circular probes and linear circularizable probes made according to the methods described herein. The present invention further provides kits comprising the circular probes and/or the linear circularizable probes of the present invention.

This application claims the benefit of U.S. Provisional Application No. 60/667,781, filed Mar. 31, 2005, the contents of which are incorporated herein by reference.

### INTRODUCTION

The present invention relates to methods for preparing linear circularizable nucleic acid probes and/or circular nucleic acid probes. Such probes may be used as the starting material for rolling circle amplification (RCA) and/or ramification-extension amplification (RAM) of nucleic acid molecules. The present invention further provides circular probes and linear circularizable probes made according to the methods described herein. The present invention further provides kits comprising the circular probes and/or the linear circularizable probes of the present invention.

### BACKGROUND OF THE INVENTION

A number of techniques have been developed recently to meet the demands for rapid and accurate detection of infectious agents, such as viruses, bacteria and fungi, and detection of normal and abnormal genes. Such techniques, which generally involve the amplification and detection (and subsequent measurement) of minute amounts of target nucleic acids (either DNA or RNA) in a test sample, include inter alia rolling circle amplification (RCA) (U.S. Pat. No. 6,855,523, incorporated herein by reference); ramification amplification methods (RAM) (U.S. Pat. Nos. 5,942,391, 6,569,647, 6,593,086 and 6,855,523, each of which are incorporated herein by reference); the polymerase chain reaction (PCR) (Saiki, et al., Science 230:1350, 1985; Saiki et al., Science 239:487, 1988; PCR Technology, Henry A. Erlich, ed., Stockton Press, 1989; Patterson et al., Science 260:976, 1993), ligase chain reaction (LCR) (Barany, Proc. Natl. Acad. Sci. USA 88:189, 1991), strand displacement amplification (SDA) (Walker et al., Nucl. Acids Res. 20:1691, 1992), Qβ replicase amplification (QβRA) (Wu et al., Proc. Natl. Acad. Sci. USA 89:11769, 1992; Lomeli et al., Clin. Chem. 35:1826, 1989) and self-sustained replication (3SR) (Guatelli et al., Proc. Natl. Acad. Sci. USA 87:1874-1878, 1990). While all of these techniques are powerful tools for the detection and identification of minute amounts of a target nucleic acid in a sample, they all suffer from various problems, which have prevented their general applicability in the clinical laboratory setting for use in routine diagnostic techniques.

RCA refers to a method for amplifying DNA that is based upon the “rolling circle” replication mechanism used by a number of single-stranded DNA bacteriophage to replicate their genomes. For RCA, a single stranded linear DNA molecule (circularizable probe, or “C” probe) is designed in a way that its terminal sequences will anneal to contiguous and complementary sequences in a “target” nucleic acid such that the 5′ and 3′ termini of the circularizable probe are brought next to one another. The ends then are joined together, by either enzymatic or chemical means, to form a covalently closed single-stranded circular DNA molecule. Subsequently, during amplification, one or more short oligonucleotide extension primers are added, along with dNTPs, an appropriate DNA polymerase, and appropriate buffer components to initiate either linear RCA or exponential RAM.

In linear RCA, a single extension primer anneals to the single-stranded DNA circular probe and the polymerase extends the primer strand around the circular probe. As the polymerase extends the primer by traveling along the circular probe, the growing DNA strand, which is complementary to the circular probe, encounters the 5′ end of the original primer, displaces it and continues the extension process. Ultimately, a long single-stranded DNA product that contains multiple tandem complementary copies of the original circular probe sequence is produced. In exponential RAM, a second extension primer anneals, not to the circular probe, but to the complementary product of the first extension primer reaction, such that a cascade of amplified products is formed. All the products of either the first or second extension primer are templates for the other primer, so that an exponential growth of product DNA occurs.

If the terminal sequences of the circularizable probe are selected to be complementary to a diagnostically useful nucleic acid sequence in a particular “target” organism or virus then circle formation and the subsequent amplified production of DNA, which is contingent on circle formation, can be used as an indicator of the presence of that target organism in a tested sample.

Critical to the success of RCA or RAM amplification is the efficient production of DNA from either pre-formed circular probes or circular probes formed as part of the assay, prior to the amplification step. Therefore, the efficiency of the RCA amplification (and therefore the sensitivity of any assay incorporating it) is critically dependent on the quality of the circular probes.

Our laboratory has observed that only a small portion of commercially synthesized circularizable probes were able to function appropriately in the RCA reaction. The cause for this deficiency is unknown. However, one could hypothesize that it is a consequence of the synthesis chemistry commonly employed by commercial DNA synthesis establishments. The synthesis chemistry typically starts with a single nucleotide phosphoramidite attached to a glass support surface (i.e., a controlled pore glass (CPG), which are beads packed into a column and used on an automated DNA synthesizer). The desired DNA sequence is “built-up” one nucleotide at a time on this starting substrate by sequential additions of the nucleotide phosphoramidites corresponding to the desired sequence, starting from the 3′ end phosphoramidite attached to the CPG bead. These phosphoramidites are chemically different in a number of respects from the nucleotides that will ultimately be part of the final product. In particular, they contain “protecting” groups on reactive side-chains, which prevent participation in addition reactions thereby producing undesirable branched molecules, rather than the desired linear product. Further, they contain “blocking” groups at the point of addition that limit each addition reaction to one nucleotide phosphoramidite per addition cycle.

During the course of a DNA synthesis, the blocking group from the 5′ terminal nucleotide phosphoramidite of the growing chain is chemically removed so that the next addition step can be performed. The groups protecting the side chains are not removed at this time. As discussed above, the added nucleotide phosphoramidite already contains the blocking group so that only one residue can be added at each step. Finally, after this cycle (i.e., addition, unblocking, addition, unblocking, etc.) is completed to produce the entire desired sequence, a different, and harsher, chemical reaction is performed to remove the protecting groups from all the nucleotide phosphoramidites in the completed DNA chain. Then, the DNA is chemically released from the CPG support and optionally is further purified by a variety of means (e.g., chromatography or polyacrylamide gel electrophoresis).

During the recent evolution of commercial DNA synthesis methods, the reagents and strategies used to make DNA have been highly optimized to raise the efficiency and lower the cost of the process. However, there remains a delicate balance between efficiency and accuracy. In particular, while it is desired that 100% of the blocking groups be removed at each cycle, the protecting groups, the nucleotide phosphoramidites themselves, and the linkage holding the 3′ phosphoramidite to the CPG support should, ideally, be 100% refractory to the unblocking chemistry. In general, this condition is largely met for shorter oligonucleotides (e.g., typical 20-25 nucleotide long PCR primers) with greater than 99.9% efficiencies of addition commonly quoted for each step. However, for longer sequences (i.e., greater than about 50 nucleotides) the consequence of the residual inefficiencies can readily be seen on the purification gel, where 1% or less of the final product often is full length. The great majority of sequences are shorter than the final desired product by varying lengths. Thus, in order to promote efficient unblocking and addition, harsher conditions must be used. However, harsher unblocking conditions lower the efficiency of retaining the protecting groups and not damaging the nucleotide phosphoramidites themselves.

It is known that for long oligonucleotides, the 3′ nucleotides that are added first are subject to most, if not all, of the subsequent addition cycles and are therefore particularly vulnerable to various forms of damage. Such damage could impede or entirely block the progress of DNA polymerase. Depurinations usually result in strand breakage during the deprotection step, yielding the short products seen on purification gels (Kwiatkowski, et al., Nucl. Acids Res. 24:4632-4638, 1996). Kwiatkowski et al. (1996, ibid) describe an alternate chemical synthesis method that attempts to ensure that long circularizable probes (i.e., “Padlock probes,” in their terminology) can be produced in good yeild with correct 5′ and 3′ ends. Similarly, Antson, et al. (Nucl. Acids Res. 28, e58: I-vi, 2000) describe a PCR-based synthesis method for small-scale production of long oligonucleotides having correct 5′ and 3′ ends. However, in both reports, shorter than full-length products are still observed. Moreover, since neither paper is concerned with the enzymatic amplification of the circularized probes, only the functionality of the 5′ and 3′ ends, necessary for circularization, is addressed.

Of more concern in the context of circular probes that are to be enzymatically amplified are full-length products which contain such polymerase impeding or blocking defects that do not result in strand scission and therefore remain in the final purified product. For example, depurination, in which the base constituent of a nucleotide is lost, but the backbone is left intact, would block DNA polymerase. Similarly, residual protecting groups in the final product also may impede or block the progression of DNA polymerase around the circular probe. Such defects are not addressed by either Anston or Kwiatkowski.

An embodiment of the current invention demonstrates that enzymatic synthesis of circularizable probes circumvents the above-mentioned problems and yields circular probes of substantially higher amplification efficiency.

The present invention relates to methods and kits for amplifying circularizable nucleic acid probes and/or single-stranded nucleic acid circular probes that may be used as the starting material for rolling circle amplification and/or ramification-extension amplification of nucleic acid molecules.

### SUMMARY OF THE INVENTION

The present invention relates to methods for preparing linear circularizable nucleic acid probes and/or circular nucleic acid probes. Such probes may be used as the starting material for rolling circle amplification (RCA) and/or ramification-extension amplification (RAM) of nucleic acid molecules. The present invention further provides circular probes and linear circularizable probes made according to the methods described herein. The present invention further provides kits comprising the circular probes and/or the linear circularizable probes of the present invention.

Embodiments of the present invention satisfy the foregoing, as well as other, needs. In accordance with one embodiment of the present invention, there is provided a method for producing circular probes comprising: contacting a first spanning oligonucleotide under conditions that allow nucleic acid hybridization between complementary sequences in the nucleic acid with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable amplification probe, wherein the circularizable amplification probe is bound on its 3′ and 5′ ends to the adjacent but not overlapping sequences in the spanning oligonucleotide; ligating the 3′ and 5′ ends of the circularizable probe with a ligating agent that joins nucleotide sequences such that a circular probe is formed; amplifying the circular probe produced via ligation by contacting the circular probe of the ligation step with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe; cleaving the single stranded DNA of repeating units with a restriction enzyme, under conditions whereby the restriction enzyme cleaves the single stranded DNA of repeating units into single unit length oligonucleotides; contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide produced from the restriction cleavage step; repeating the ligating, extension and restriction enzyme cleavage steps; contacting the first spanning oligonucleotide under conditions that allow nucleic acid hybridization between complementary sequences in the first spanning oligonucleotide with at least one unit length oligonucleotide produced from the final restriction enzyme cleavage step; and ligating the 3′ and 5′ ends of the unit length oligonucleotide with a ligating agent that joins nucleotide sequences such that a circular probe is formed, which is an exact copy of the circular probe that resulted from the first ligation step, except that it is enzymatically produced, rather than chemically synthesized.

Another embodiment of the present invention includes as amplification kit comprising: at least one first spanning oligonucleotide; at least one circularizable amplification probe; at least one extension primer; and at least one second spanning oligonucleotide.

Yet another embodiment of the present invention includes a method for producing a linear circularizable probe comprising: contacting a first spanning oligonucleotide under conditions that allow nucleic acid hybridization between complementary sequences in the nucleic acid with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable amplification probe, wherein the circularizable amplification probe is bound on its 3′ and 5′ ends to the adjacent but not overlapping sequences in the spanning oligonucleotide; ligating the 3′ and 5′ ends of the circularizable probe with a ligating agent that joins nucleotide sequences such that a circular probe is formed; amplifying the circular probe of the ligation step by contacting the circular probe produced via ligation with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe; cleaving the single stranded DNA of repeating units with a restriction enzyme, under conditions whereby the restriction enzyme cleaves the single stranded DNA of repeating units into single unit length oligonucleotides; contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide produced from the restriction enzyme cleavage step; repeating the ligation, extension, and restriction enzyme cleavage steps such that a linear circularizable probe is formed, which is an exact copy of the circularizable probe from the beginning of the process, except that it is enzymatically produced, rather than chemically synthesized. It is readily appreciated by the skilled artisan that the probes of the present invention may be used in standard amplification methods including, but not limited to, RCA, RAM, PCR and HSAM.

Another embodiment of the present invention includes a single stranded circular probe comprising regions that are complementary to adjacent but not overlapping sequences of a spanning oligonucleotide, wherein the circular probe is made according to the method described above.

Another embodiment of the present invention includes a single stranded linear circularizable probe comprising 3′ and 5′ regions that are complementary to adjacent but not overlapping sequences of a spanning oligonucleotide, wherein the circularizable probe is made according to the method of claim described above.

### BRIEF DESCRIPTION OF THE DRAWINGS

FIG. 1 is a schematic diagram of amplification of a circularized probe by primer-extension/displacement and PCR.

FIG. 2 is a schematic diagram of HSAM using a circular target probe and three circular signal probes. AB, CD and EF indicate nucleotide sequences in the linker regions that are complementary to the 3′ and 5′ nucleotide sequences of a circular signal probe. AB′, CD′ and EF′ indicate the 3′ and 5′ nucleotide sequences of the signal probes that have been juxtaposed by binding to the complementary sequences of the linker regions of another circular signal probe.

FIG. 3 is a schematic diagram of HSAM utilizing a circular target probe and linear signal probes.

FIG. 4 is a schematic diagram of amplification of a circularized probe by the ramification-extension amplification method (RAM).

FIG. 5 shows an ethidium bromide stained preparative gel provided by the manufacturer of the Stachyl39 circularizable probe before and after purification.

FIG. 6 shows the DNA patterns from 4 preparative polyacrylamide gels that demonstrate the various ³³P-labeled DNA products and intermediates generated during the process of making enzymatic plus strand Stachyl39 circular probes.

FIG. 7 is a preparative polyacrylamide gel loaded with ligated ³³P-labeled gel purified unit length plus strand Stachyl39 DNA.

FIG. 8 is a graphic representation of the rates of synthesis of product DNA during the two RCA reactions involved in the process of generating enzymatically produced circular probes.

FIG. 9 is a graphic representation of the increase in the number of unit copies of Stachyl39 DNA over time where several input levels of synthetic circular templates are compared with those produced enzymatically.

FIG. 10 is a graphic representation that the chemically and enzymatically synthesized circular probes replicate in exponential RAM assays with virtually identical kinetics.

### DETAILED DESCRIPTION OF THE INVENTION

The present invention relates to methods for preparing linear circularizable nucleic acid probes and/or circular nucleic acid probes. Such probes may be used as the starting material for rolling circle amplification (RCA) and/or ramification-extension amplification (RAM) of nucleic acid molecules. The present invention further provides circular probes and linear circularizable probes made according to the methods described herein. The present invention further provides kits comprising the circular probes and/or the linear circularizable probes of the present invention.

During the course of detailed investigations on the efficiency of the RCA reaction it was discovered that only a small proportion of single stranded DNA circular probes produced by standard means (i.e., chemically synthesized), were able to participate in the RCA or RAM reactions. Moreover, it was discovered that this was true for multiple probe sequences manufactured by multiple commercial DNA synthesis establishments. Therefore, the defects causing replication deficiencies are not sequence specific or vendor specific. The range of efficiencies observed ranged from a high of about 10% (i.e., 10% of chemically synthesized circularizable probes could actually replicate) to substantially less than 1%. Substantial variation was observed even in different synthesis lots of the same DNA sequence of individual vendors. The deficiency appears to be related to a poorly controlled variable during standard phosphoramidite synthesis. Also, since all tested probes were gel purified, the defective probe sequences cannot simply be separated from non-defective product by size-exclusion.

It is evident that very few defects in the DNA chain are necessary to produce the observed loss of efficiency seen in RCA amplification. In a 100 nucleotide long DNA circularizable probe, a probability of 1% of a defect occurring at any position implies that the fraction of circularizable probes having at least one defect would be 1-0.99¹⁰⁰. Therefore, 63% of the circularizable probes would be defective. A probability of 0.1% defect occurrence at any nucleotide in a 100 nucleotide circularizable probe would result in the fraction of defective circularizable probes being 1-0.999¹⁰⁰, or 9.5%. Our observation suggest that between 90 and 99% of 100 nucleotide long circular probes contain DNA polymerase stopping defects, which would correspond to a defect rate of between 2.3% to 4.5% per nucleotide.

The single, full length, ligation-dependent circularizable probe (i.e., C-probe), as utilized in the method, affords greater efficiency of the detection and amplification of the target nucleic acid sequence. Due to the helical nature of double-stranded nucleic acid molecules, circularized probes are wound around the target nucleic acid strand. As a result of the ligation step, the probe may be irreversibly bound to the target molecule by means of catenation. This results in immobilization of the probe on the target molecule, forming a hybrid molecule that is substantially resistant to stringent washing conditions. This results in significant reduction of non-specific signals during the assay, lower background noise and an increase in the specificity of the assay.

In another embodiment of the present invention, there is provided a method of enzymatically synthesizing circular probes that overcomes the poor performance problems associated with the chemically synthesized, via phosphoramidite chemistry, circular probes. A chemically synthesized, single-stranded linear DNA oligonucleotide (designated a “plus strand”) is incubated with a short oligonucleotide (i.e., spanning oligonucleotide), complementary to and spanning the 5′ and 3′ ends of the plus strand linear oligonucleotide, under conditions that promote hybridization between the complementary nucleotides. DNA ligase is added to the complex to form a covalently closed circular probe. A suitable DNA ligase can be Taq DNA ligase or T4 DNA ligase.

The plus strand circular probes are then subjected to single primer RCA. Briefly, the circular probe is incubated with: an extension primer, which is complementary to sequences in the circular probe and which may or may not be identical to the spanning oligonucleotide, a DNA polymerase with strand displacement activity, such as Φ29 DNA polymerase or Bst DNA polymerase, and dNTPs under conditions that promote the formation of a minus strand multimeric linear DNA reaction product comprising up to 10,000 or more complementary copies of the circular probe.

The multimeric reaction product is then cleaved into “unit length” fragments by the addition of an appropriate restriction enzyme. The unit length fragments (minus strand) are then purified via polyacrylamide gel electrophoresis, excision and elution of the appropriate-sized band, as is well know by the skilled artisan.

Minus strand circular probes are then prepared from the purified unit length fragments via ligation as described above. Alternatively, one may begin the enzymatic synthesis reaction with chemically synthesized minus strand circular probes.

Finally, one more round of RCA, restriction to unit length size, gel purification, circularization, and gel purification is performed. The final product consists of highly purified quantitated plus strand circular probes identical in sequence to the starting circular probes except they were produced by DNA polymerase rather than on a DNA synthesis machine by phosphoramidite chemistry.

It will be evident to one of ordinary skill in the art that the foregoing method offers advantages by imposing a number of selective steps on the original “population” of chemically synthesized linear probe sequences. First, only sequences containing 5′ and 3′ terminal sequences complementary to the spanning oligonucleotide will circularize upon ligation. Other sequences, not able to circularize will be eliminated by the gel purification step as they will not migrate with the correctly sized circularized product. Circularization induces a significantly larger mobility shift than would be seen, for example, between full length linear oligonucleotides and so-called “n−1” oligonucleotides (i.e., sequences 1 nucleotide shorter than full length). Second, only sequences which are able to replicate by the chosen DNA polymerase, for example Φ29 DNA polymerase, will be represented in the product of the first or second RCA amplification. This necessitates the correct annealing of an oligonucleotide primer to the circular probe, thus selecting for the presence of the correct complementary sequence in the circular probe, and the ability of the polymerase to traverse around the whole of the circular probe multiple times unimpeded. The ability of the polymerase to traverse around the circular probe selects for non-defective circular probes from the chemically synthesized population without any requirement for knowing the nature of such chemical defects. In the enzymatic synthesis method described above and illustrated by Example 2, there are at least 4 oligonucleotide binding events imposed: two ligation/circularization steps and two priming steps. Therefore, at least four oligonucleotide-sized segments of the final product can be ensured to have the correct sequence. In addition, there are two restriction endonuclease cleavage steps that also can be used to impose a selective requirement for at least two more oligonucleotide-length sequences upon the final product. Thus, sequences containing at least all functionally important sequence elements of the final product oligonucleotide probe can be selectively “extracted” from the original heterogeneous chemically synthesized population.

Circular probes with improved replication properties, such as those produced by the enzymatic process described above, could be useful in a variety of contexts as elements of a signal generating system in diagnostic assays as described herein. For example, the circular probes could be hybridized directly to target nucleic acids, and then amplified and detected to signify the presence of that target nucleic acid in a sample. Circular probes could also be hybridized directly to target nucleic acids and detected via HSAM, as described herein. Alternatively, they could be attached to a target nucleic acid through an intermediate probe that hybridized both to target nucleic acid and the circular probe, and similarly amplified and detected. Alternatively, they could be directly or indirectly linked to antibodies, proteins, or aptimers that can bind to various non-nucleic acid targets (e.g. proteins) and thus used as signal generating moieties in a wide variety of diagnostic assays.

In yet another embodiment of the present invention, it also is desirable to utilize such circularizable probes in ligation-based assays, such that the 5′ and 3′ ends of a linear probe are brought together by, for example, a specific nucleic acid target sequence. In this format, the 5′ and 3′ ends of the linear probe are covalently joined together, forming a circle, in a manner that can be made highly dependent upon the presence of the correct target sequence. The method described above and in Example 2, relies on a restriction endonuclease to generate the termini of the linear circularizable probe intermediate, and therefore, the 5′ and 3′ termini are necessarily defined by the recognition sequence of a particular restriction endonuclease. In Example 2, PvuII restriction endonuclease was used and the termini of the final linear intermediate therefore have the sequence 5′-CTG . . . NNN . . . CAG-3′, where NNN represents the chosen nucleotide sequence between the termini. While there are a wide variety of restriction endonucleases, with a wide variety of recognition sequences available, it nevertheless would be advantageous to completely relieve this restriction of possible enzymatically produced sequences to permit the widest possible range of prospective target sequences to be utilized.

Following the procedure described in Example 2, except that the starting chemically synthesized oligonucleotide has an additional sequence comprising the recognition sequence for a Type IIB restriction endonuclease (e.g., BsaXI) and flanking sequences such that, upon incubating a double stranded version of the sequence with the Type IIB restriction endonuclease, the additional sequence is precisely cleaved at the termini of the flanking sequences. This oligonucleotide can be processed exactly as described in Example 2, except that the multimeric plus strand amplification product of the second RCA reaction is reduced to unit length pieces using the said Type IIB restriction endonuclease. Type IIB restriction endonucleases have the useful property that their recognition sequence and restriction site are not the same. For example, BsaXI, has the recognition/cutting sequence 5′- . . . ^{V}(N)₉AC(N)₅CTCC(N)₁₀^{V} . . . -3′. The enzyme recognizes the central AC(N)₅CTTCC sequence and cuts 9 nucleotides upstream and 10 nucleotides downstream of its recognition sequence without regard to the actual sequence at the cut site. Therefore, inserting this sequence into a multimeric single stranded RCA product, adding a complementary oligonucleotide to render this portion of the multimer double stranded, and incubating with BsaXI under appropriate conditions, will result in the production of unit length circularizable oligonucleotides identical to those of Example 2, except that the 5′ and 3′ terminal sequences can be of any arbitrarily chosen sequence.

In another embodiment of the present invention, an existing circularizable probe can be amplified using a PCR reaction with the circularizable probe as template, and primers that have 5′ extensions designed to create a specific Type IIB restriction enzyme recognition site when one strand of the PCR product is circularized. The advantage of this scheme over a total synthesis of circularizable probes is that the synthesis is done in fewer steps with lower cost.

By design, only one strand of the double-stranded PCR product is desired for the subsequent step of the process. The PCR reaction can be biased for production of the desired strand by adjusting the ratio of the two PCR primers (See Sanchez, J. A., et al., Proc Natl Acad Sci USA 101(7): 1933-8, 2004).

After the PCR reaction, the desired strand is circularized by ligation on a short template oligonucleotide. The resulting circular probe is a template for a RCA reaction that creates multiple concatamerized copies of a DNA strand that is the complement of the template circular probe. An oligonucleotide sequence complementary to the concatamerized product and that creates the desired Type IIB restriction endonuclease site is allowed to anneal to the concatamer, and the chosen Type IIB restriction enzyme is added to create unit-length circularizable probes with the desired end-structures.

One of the desirable features of the circularizable probe detection system is that a single oligonucleotide primer can be used to amplify circularized probes that detect different target regions. This is possible because the amplification is initiated in a generic internal region of the linear circularizable probe, while the target region specificity is due to the target-specific end sequences of the circularizable probe. The PCR scheme should allow the synthesis of any desired target region on a given generic region.

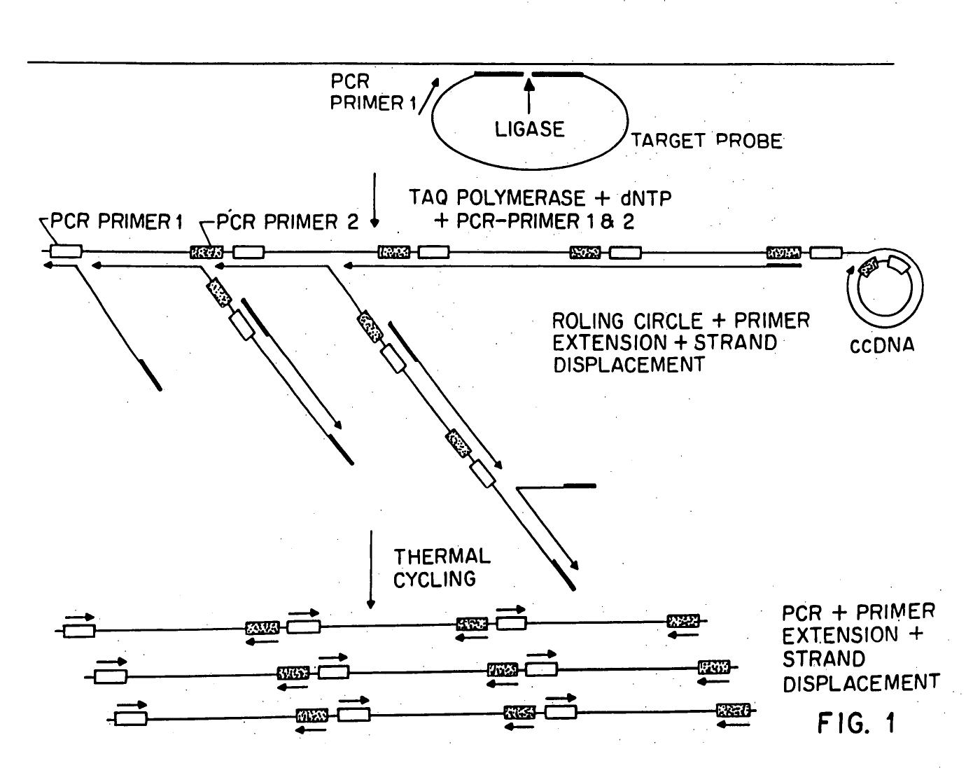

The circularized probe can also be amplified and detected by the generation of a large polymer. The polymer is generated through the rolling circle extension (i.e., rolling circle amplification (RCA)) of primer 1 (e.g., extension primer 1) along the circularized probe and displacement of the downstream sequence. This step produces a single stranded DNA containing multiple units that serves as a template for subsequent PCR, as depicted in FIG. 1. As shown therein, primer 2 (e.g., extension primer 2) can bind to the single stranded DNA polymer and extend simultaneously, resulting in displacement of downstream primers by upstream primers. By using both primer-extension/displacement and PCR, more detectable product is produced with the same number of cycles.

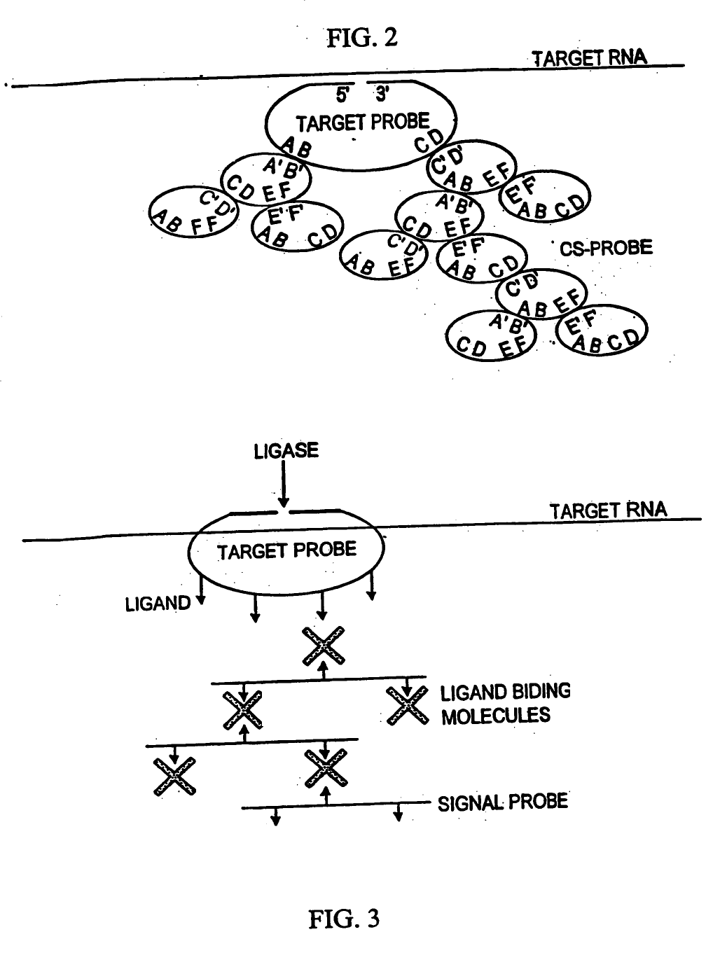

The circularized probe may also be detected by a modification of the HSAM assay. In this method, depicted in FIG. 2, the circularizable probe (referred to as a Target Probe in FIG. 2) contains, as described hereinabove, 3′- and 5′ regions that are complementary to adjacent regions of the target nucleic acid. The circularizable probes further contain a non-complementary, or generic linker regions. In the present signal amplification method, the linker region of the circularizable probe contains at least one pair of adjacent regions that are complementary to the 3′ and 5′ regions of a first generic circularizable signal probe (CS-probe). The first CS-probe contains, in its 3′ and 5′ regions, sequences that are complementary to the adjacent regions of the linker region of the circularizable probe. Binding of the circularizable probe to the target nucleic acid, followed by ligation, results in a covalently linked circular probe having a region in the linker available for binding to the 3′ and 5′ ends of a first CS-probe. The addition of the first CS-probe results in binding of its 3′ and 5′ regions to the complementary regions of the linker of the circular probe. The 3′ and 5′ regions of the CS-probe are joined by the ligating agent to form a closed circular CS-probe bound to the closed circular probe. The first CS-probe further contains a linker region containing at least one pair of adjacent contiguous regions designed to be complementary to the 3′ and 5′ regions of a second CS-probe.

The second CS-probe contains, in its 3′ and 5′ regions, sequences that are complementary to the adjacent regions of the linker region of the first CS-probe. The addition of the second CS-probe results in binding of its 3′ and 5′ regions to the complementary regions of the linker of the first CS-probe. The 3′ and 5′ regions of the second CS-probe are joined by the ligating agent to form a closed circular CS-probe, which is in turn bound to the closed circular probe.

By performing the above-described method with a multiplicity of CS-probes having multiple pairs of complementary regions, a large cluster of chained molecules is formed on the target nucleic acid. In another embodiment, three CS-probes are utilized. In addition to the 3′ and 5′ regions, each of the CS-probes has one pair of complementary regions that are complementary to the 3′ and 5′ regions of a second CS-probe, and another pair of complementary regions that are complementary to the 3′ and 5′ regions of the third CS-probe. By utilizing these “trivalent” CS-probes in the method of the invention, a cluster of chained molecules as depicted in FIG. 2 is produced.

Following extensive washing to remove non-specific chain reactions that are unlinked to the target, the target nucleic acid is then detected by detecting the cluster of chained molecules. The chained molecules can be easily detected by digesting the complex with a restriction endonuclease for which the recognition sequence has been uniquely incorporated into the linker region of each CS-probe. Restriction endonuclease digestion results in a linearized monomer that can be visualized on a polyacrylamide gel. Other methods of detection can be effected by incorporating a detectable molecule into the CS-probe, for example digoxigenin, biotin, or a fluorescent molecule, and detecting with anti-digoxinin, streptavidin, or fluorescence detection. Latex agglutination, as described for example by Essers et al, J. Clin. Microbiol., 12, 641, 1980, may also be used. Such nucleic acid detection methods are known to one of ordinary skill in the art.

In another embodiment, the circularized probe may also be detected by another modification of the HSAM assay. In this method, depicted in FIG. 3, ligand molecules are incorporated into the linker region of the circularizable probe, for example during probe synthesis. The HSAM assay is then performed as described hereinabove and depicted in FIG. 3 by adding ligand binding molecules and signal probes to form a large complex, washing, and then detecting the complex. Nucleic acid detection methods are known to those of ordinary skill in the art and include, for example, latex agglutination as described by Essers, et al., J. Clin. Microbiol., 12:641, 1980. The use of circularizable probes in conjunction with HSAM is particularly useful for in situ hybridization.

The present methods may be used with routine clinical samples obtained for testing purposes by a clinical diagnostic laboratory. Clinical samples that may be used in the present methods include, inter alia, whole blood, separated white blood cells, sputum, urine, tissue biopsies, throat swabbings and the like, i.e., any patient sample normally sent to a clinical laboratory for analysis.

The complex can be detected by methods known in the art and suitable for the selected ligand and ligand binding moiety. For example, when the ligand binding moiety is streptavidin, it can be detected by immunoassay with streptavidin antibodies. Alternately, the ligand binding molecule may be utilized in the present method as a conjugate that is easily detectable. For example, the ligand may be conjugated with a fluorochrome or with an enzyme that is detectable by an enzyme-linked chromogenic assay, such as alkaline phosphatase or horseradish peroxidase. For example, the ligand binding molecule may be alkaline phosphatase-conjugated streptavidin, which may be detected by addition of a chromogenic alkaline phosphatase substrate, e.g., nitroblue tetrazolium chloride.

Any suitable technique for detecting the signal generating moiety may be utilized. Such techniques include scintillation counting (for ³²P) and chromogenic or fluorogenic detection methods as known in the art. For example, suitable detection methods may be found, inter alia, in Sambrook et al., Molecular Cloning—A Laboratory Manual, 2d Edit., Cold Spring Harbor Laboratory, 1989, in Methods in Enzymology, Volume 152, Academic Press, 1987, or Wu et al., Recombinant DNA Methodology, Academic Press, 1989.

The term “ligand” as used herein refers to any component that has an affinity for another component termed here as “ligand binding moiety.” The binding of the ligand to the ligand binding moiety forms an affinity pair between the two components. For example, such affinity pairs include, inter alia, biotin with avidin/streptavidin, antigens or haptens with antibodies, heavy metal derivatives with thiogroups, various polynucleotides such as homopolynucleotides as poly dG with poly dC, poly dA with poly dT and poly dA with poly U. Any component pairs with strong affinity for each other can be used as the affinity pair, ligand-ligand binding moiety. Suitable affinity pairs are also found among ligands and conjugates used in immuno-logical methods.

Ligating agents are well known in the art. Examples of such ligating agents include, but are not limited to, an enzyme, e.g., a DNA or RNA ligase, or chemical joining agents, e.g., cyanogen bromide or a carbodiimide (Sokolova et al., FEBS Lett. 232:153-155, 1988).

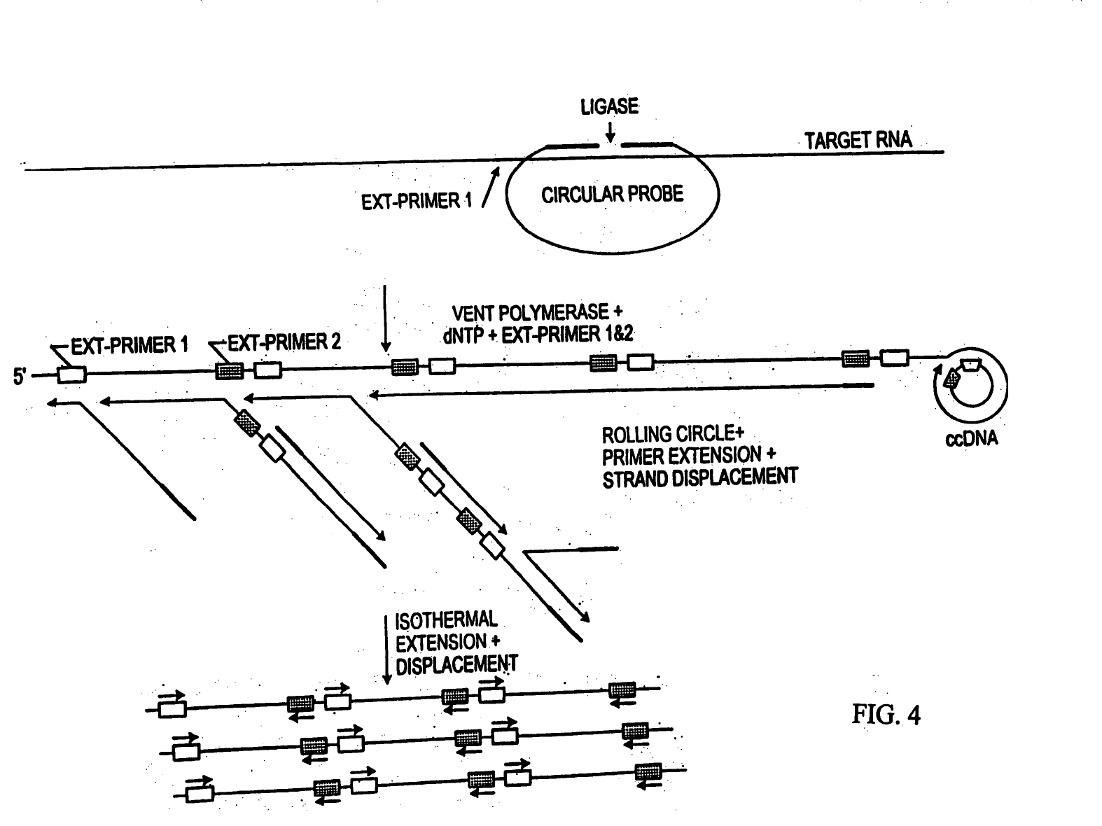

In embodiments of the present invention utilizing a ligation dependent circularizable probe, the resulting circular molecule may be conveniently amplified by the ramification-extension amplification method (RAM), as depicted in FIG. 4. Amplification of the circularized probe by RAM adds still further advantages to the methods of the present invention by allowing up to a million-fold amplification of the circularized probe under isothermal conditions. RAM is illustrated in FIG. 4.

The single, full length, ligation dependent circularizable probe useful for RAM contains regions at its 3′ and 5′ termini that are hybridizable to adjacent but not overlapping regions of the target molecule. The circularizable probe is designed to contain a 5′ region that is complementary to and hybridizable to a portion of the target nucleic acid, and a 3′ region that is complementary to and hybridizable to a portion of the target nucleic acid adjacent to the portion of the target that is complementary to the 5′ region of the probe. The complementary 5′ and 3′ regions of the circularizable probe may each be from about 20 to about 35 nucleotides in length. In another embodiment, the 5′ and 3′ regions of the circularizable probe are about 25 nucleotides in length. The circularizable probe further contains a region designated as the linker region. In yet another embodiment the linker region is from about 30 to about 60 nucleotides in length. The linker region is composed of a generic sequence that is neither complementary nor hybridizable to the target sequence.

The circularizable probe suitable for amplification by RAM is utilized in the present method with one or more capture/amplification probes, as described hereinabove. When the circularizable probe hybridizes with the target nucleic acid, its 5′ and 3′ termini become juxtaposed. Ligation with a linking agent results in the formation of a closed circular molecule (e.g., the circular probe).

Amplification of the closed circular molecule is effected by adding a first extension primer (Ext-primer 1) to the reaction. Ext-primer 1 is complementary to and hybridizable to a portion of the linker region of the circular probe, and can be from about 15 to about 30 nucleotides in length. Ext-primer 1 is extended by adding sufficient concentrations of dNTPs and a DNA polymerase to extend the primer around the closed circular molecule. After one revolution of the circle, i.e., when the DNA polymerase reaches the Ext-primer 1 binding site, the polymerase displaces the primer and its extended sequence. The polymerase thus continuously “rolls over” the closed circular probe to produce a long single strand DNA, as shown in FIG. 4.

The polymerase useful for amplification of the circularized probe by RAM may be any polymerase that lacks 3′→5′ exonuclease activity, that has strand displacement activity, and that is capable of primer extension of at least about 1000 bases. (Exo-)Klenow fragment of DNA polymerase, Thermococcus litoralis DNA polymerase (Vent (exo-)) DNA polymerase, New England Biolabs), Bacillus stearothermophilus DNA polymerase (Bst DNA polymerase) and phi29 polymerase (Blanco et al., Proc. Natl. Acad. Sci. USA, 91:12198, 1994) are examples of such polymerases. Thermus aquaticus (Taq) DNA polymerase is also useful in accordance with the present invention. Contrary to reports in the literature, it has been found in accordance with the present invention that Taq DNA polymerase has strand displacement activity.

Extension of Ext-primer 1 (i.e., RCA) by the polymerase results in a long single stranded DNA molecule of repeating units having a sequence complementary to the sequence of the circular probe. The single stranded DNA may be up to 10 Kb, and for example may contain from about 20 to about 100 units, with each unit equal in length to the length of the circularizable probe, for example about 100 bases. As an alternative to RAM, detection may be performed at this RCA step if the target is abundant or the single stranded DNA is long. For example, the long single stranded DNA may be detected at this stage by visualizing the resulting product as a large molecule on a polyacrylamide gel.

In the next step of amplification by RAM, a second extension primer (Ext-primer 2) is added. Ext-primer 2 may be about 15 to about 30 nucleotides in length. Ext-primer 2 is identical to a portion of the linker region that does not overlap the portion of the linker region to which Ext-primer 1 is complementary. Thus each repeating unit of the long single stranded DNA contains a binding site to which Ext-primer 2 hybridizes. Multiple copies of the Ext-primer 2 thus bind to the long single stranded DNA, as depicted in FIG. 4, and are extended by the DNA polymerase. The primer extension products displace downstream primers with their corresponding extension products to produce multiple displaced single stranded DNA molecules, as shown in FIG. 4. The displaced single strands contain binding sites for Ext-primer 1 and thus serve as templates for further primer extension reactions to produce the multiple ramification molecule shown in FIG. 4. The reaction comes to an end when all DNA becomes double stranded.

The DNA amplified by RAM is then detected by methods known in the art for detection of DNA. Because RAM results in exponential amplification, the resulting large quantities of DNA can be conveniently detected, for example by gel electrophoresis and visualization for example with ethidium bromide. Because the RAM extension products differ in size depending upon the number of units extended from the closed circular DNA, the RAM products appear as a smear or ladder when electrophoresed. In another embodiment, the circularizable probe is designed to contain a unique restriction site, and the RAM products are digested with the corresponding restriction endonuclease to provide a large amount of a single sized fragment of one unit length i.e., the length of the circularizable probe. The fragment can be easily detected by gel electrophoresis as a single band. Alternatively, a ligand such as biotin or digoxigenin can be incorporated during primer extension and the ligand-labeled single stranded product can be detected as described hereinabove.

The RAM extension products can be detected by other methods known in the art, including, for example, ELISA, as described hereinabove for detection of PCR products, or by real time fluorescence assay (e.g., incorporating Sybr Green intercalating dye into the reaction).

Reagents for use in practicing the present invention may be provided individually or may be packaged in kit form. For example, kits might be prepared comprising one or more first spanning oligonucleotides, one or more circularizable probes, one or more first extension primers, one or more second spanning oligonucleotides and one or more second extension primers. Such kits may also comprise packaged combinations of appropriate reagents required for ligation (e.g., DNA ligase) and, possibly, amplification (e.g., an appropriate DNA polymerase) may be included.

The arrangement of the reagents within containers of the kit will depend on the specific reagents involved. Each reagent can be packaged in an individual container, but various combinations may also be possible.

The present invention is illustrated with the following examples, which are not intended to limit the scope of the invention.

### EXAMPLE 1

### Production of Chemically Synthesized Single-Stranded DNA Circular Probes

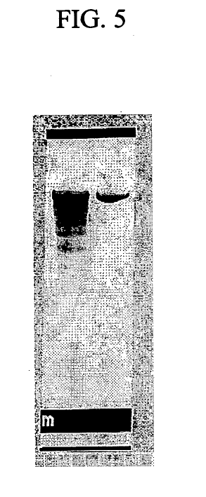

Linear DNAs ranging from 88 to 124 nucleotides in length were prepared by standard phosphoramidite chemistry by Gene Link, Inc and purified by polyacrylamide gel electrophoresis. FIG. 5 is a picture of an ethidium bromide stained gel of a 110 nucleotide long sequence (Stachyl39) before and after gel purification by the manufacturer. ³³P-labeled phosphate was added to 5′ ends via standard polynucleotide kinase reactions. Plus-strand circular DNA probes were formed by annealing a short oligonucleotide (i.e., spanning oligonucleotide) which has the following sequence, 5′-CACTCAGAGA ATACTGAAAA AAACACAAGA GT-3′ (SEQ ID NO. 1), and is complementary to and spanning the 5′ and 3′ ends. Then the covalently closed circular probes were formed by ligation of the ends with Taq DNA ligase. Exonuclease digestion of uncircularized molecules and gel purification of the ligation products yielded pure preparations of covalently-closed single-stranded DNA circular probes. Quantitation of the physical number of circular probes in the final preparations was achieved by counting the radioactivity in the purified circular probe preparation that, in conjunction with the known specific activity of the labeled phosphate, permits an accurate determination of the number and concentration of circular probes present. Spectrophotometric measurement was also be used when sufficient material was available.

Below are the C-probe sequences that were synthesized and tested for circularization and replication competence.

| | |
|---|---|
| Sal: | |
| 5′ GCGCCTTTCC AGACGCTTAC CAAGAGCAAC | (SEQ ID NO. 2) |
| TACACGAATT CTCGATTAGG TTACTGCGAT | |
| TAGCACAAGC GCTGTCACCC TGTATCGC 3′ | |
| Chlamy: | |
| 5′ GGTTTTGTCT TCGTAACTCG CTCCGGATGT | (SEQ ID NO. 3) |
| CTGTGTATCT GCTAACCAAG AGCAACTACA | |
| CGAATTCTC GATTAGGTTA CTGCGATTAG | |
| CACAAGCTCT ACAAGAGTAC ATCGGTCAAC | |
| GAAGA 3′ | |
| Stachy139: | |
| 5′ AGTATTCTCT GAGTGGCAAA CGCAATGAAG | (SEQ ID NO. 4) |
| CTTGTCCTAG TGTGTCAGTC GCACGCTTAC | |
| CAAGAGCAAC TACACGAACA GCTGTGACCC | |
| CAAACTCTTG TGTTTTTTTC 3+ | |
| Stachy154: | |
| 5′ AGTATTCTCT GAGTGGCAAA CGCAATGAAG | (SEQ ID NO. 5) |
| CTTGTCCTAG TGTGTCAGTC GCACGCTTACC | |
| AAGAGCAACT ACACGAACAG CTGTTGTTTT | |
| TTTC ′3 | |

### EXAMPLE 2

### Selection of Active Circular Probes by Enzymatic Synthesis

Starting with chemically synthesized (plus strand) Stachyl39 circular probes, as described in Example 1, single primer RCA reactions were performed using Φ 29 DNA polymerase and alpha ³³P-dATP to label the multimeric linear reaction products. Aliquots were withdrawn and time points of 0, 2, 4 and 20 hours were spotted onto DE81 filters for quantitation of product formation. At the 4 hour time point approximately 2,500 complementary copies were synthesized for each circular probe added to the reaction at time point 0 hours. Subsequently, the multimeric product material was cleaved into “unit length” fragments by the addition of restriction enzyme PvuII and a calculated 3-fold molar excess, with respect to the product, of a short oligonucleotide (i.e., PvuII(+) (ACACGAACAGCTGTGACCC) (SEQ ID NO. 6), 19 nucleotides) that was complementary to the replicated sequences and included a PvuII recognition/restriction sequence. These unit length fragments, corresponding to the complement (i.e., minus strand) of the original circular probe sequence (i.e., plus strand), were then purified from polyacrylamide gels by electrophoresis, excision, and elution of the appropriate-sized band. From this linear DNA, minus strand circular probes were prepared via ligation as in Example 1, except that the spanning oligonucleotide was PvuII(+). These minus strand circular probes were purified from polyacrylamide gels as described above. Finally, one more round of RCA, using PvuII(+) as primer, restriction to unit length size, gel purification, circularization, and gel purification were performed. The second round of RCA using the minus strand circular probes as templates showed increased template activity as indicated by the rate of DNA synthesis and final mass of product made. The final product consisted of highly purified quantitated plus strand circular probes identical in sequence to the starting circular probes except that they were produced by Φ 29 DNA polymerase rather than on a DNA synthesis machine by phosphoramidite chemistry.

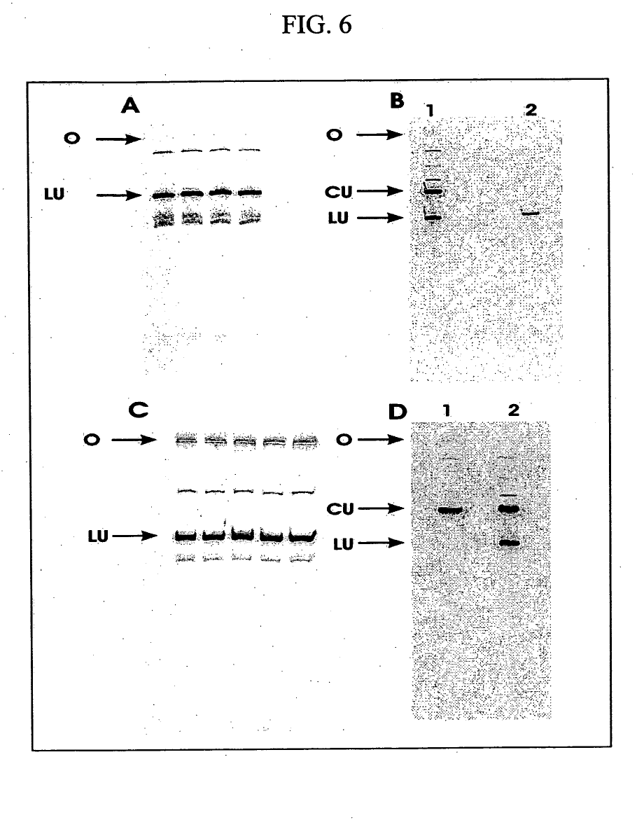

FIG. 6 shows the DNA patterns from 4 preparative polyacrylamide gels that demonstrate the various ³³P-labeled DNA products and intermediates generated during the process of making enzymatic plus strand Stachyl39 circular probes, where “O” indicates the gel origins and “CU” and “LU” represent the positions of the closed circular units and linear units, respectively. Gel A represents 4 lanes loaded with the PvuII restricted minus strand DNA derived from a Φ 29 DNA polymerase RCA amplification on synthetic oligonucleotide circular probes. Lane 1 in gel B shows the result of circularizing the linear unit eluted from gel A and Lane 2 is the unligated control. Gel C corresponds to 5 lanes loaded with the PvuII restricted plus strand DNA resulting from a Φ 29 DNA polymerase RCA amplification on enzymatic (−) strand circular probes eluted from gel B. The plus strand circular probes, formed by ligating the linear unit DNA recovered from gel C, can be seen in gel D. In Gel D, Lane 1, the loaded material was first digested with exonuclease I and III and the material in Lane 2 was untreated prior to loading.

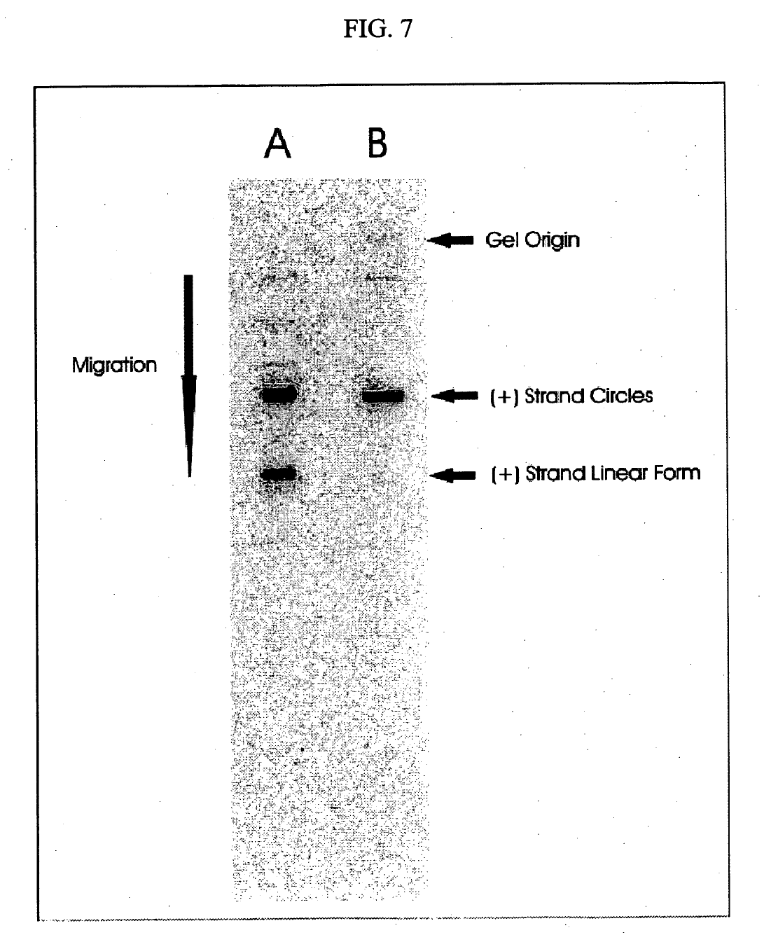

FIG. 7 is a preparative polyacrylamide gel loaded with ligated ³³P-labeled gel purified unit length plus strand Stachyl39 DNA. Lane A demonstrates the conversion of the unit length single-stranded DNA circularizable probes into slower migrating circular probe forms after ligation. Lane B is the same material as in Lane A which has been subjected to an additional exonuclease digestion step demonstrating the exonuculease resistant circular form.

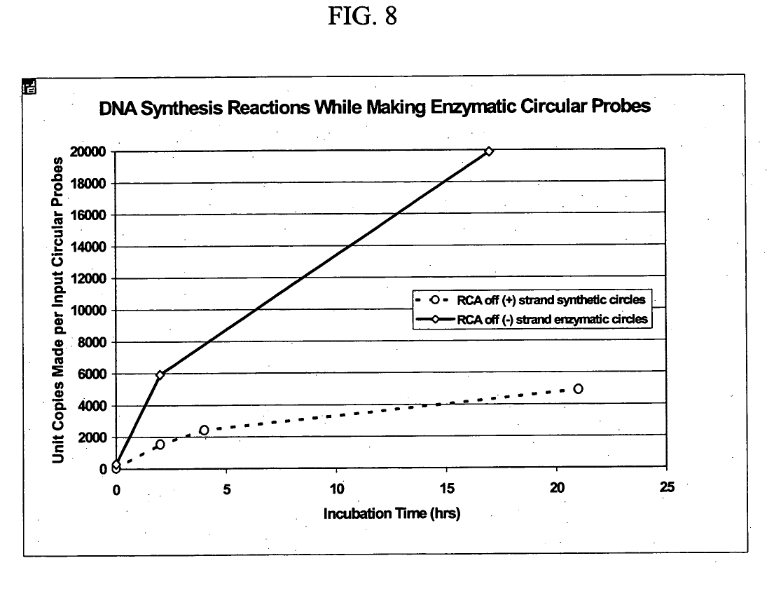

FIG. 8 depicts the rates of synthesis of product DNA during the two RCA reactions involved in the process of generating enzymatically produced circular probes. Clearly, at any time point in the reaction, amplification of enzymatically produced circular probes by phi29 DNA polymerase yields significantly more product DNA than equivalent numbers of the chemically synthesized circular probes.

### EXAMPLE 3

### Comparison of Chemically Synthesized and Enzymatically Produced Circular Probes by Single Primer RCA

Approximately equal numbers of chemically synthesized (see Example 1) and enzymatically produced (see Example 2) circular probes were used to initiate single primer RCA reactions using Φ 29 DNA polymerase and alpha ³³P-dATP to label the multimeric linear reaction products. Aliquots were withdrawn at time points of 0, 1, 2, 4, and 19 hours and spotted onto DE81 filters for quantitation of product formation.

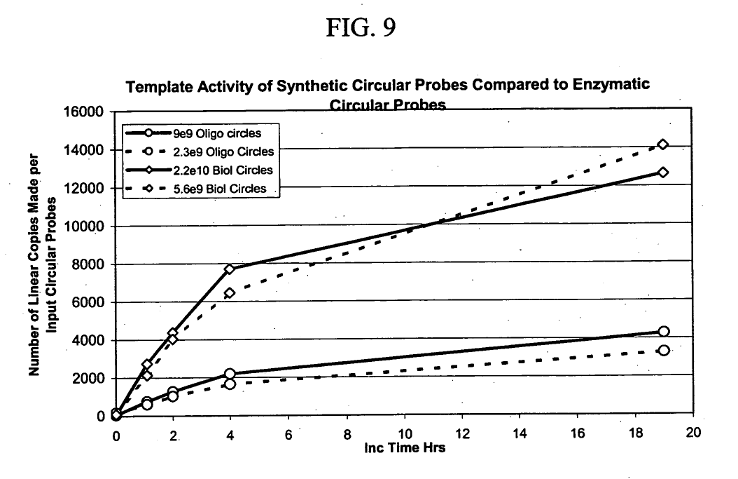

FIG. 9 represents the increase in the number of unit copies of Stachyl39 DNA over time where several input levels of synthetic circular templates are compared with those produces enzymatically. The enzymatically produced circular probes yielded about 3- to 5-fold greater amounts of product than the chemically synthesized circular probes.

### EXAMPLE 4

### Kinetic Analysis of the Fraction of Replication Competant Circular Probes in Various Circle Probe Preparations

Dilution series of chemically and enzymatically synthesized Stachyl39 circular probe preparations were made ranging from 10⁶ circular probes to less than one (1) circular probe per amplification reaction. These dilutions were each subjected to replicate exponential RCA assays (RAM assays) in which product formation was monitored in real time in a Bio-Rad real time analysis instrument. Circular probes were added to a RAM reaction mix containing 1× ThermoPol Buffer (New England BioLabs: 20 mM Tris-HCl, 10 mM KCl, 10 mM (NH₄)₂SO₄, 2 mM MgSO₄, 0.1% Triton X-100, pH8.8), 0.2 mM each dNTPs, 0.13 U/μL Bst DNA polymerase (New England BioLabs), 5% (v/v) DMSO, 40 mM NaCl, 1.25 uM Forward Primer, 0.5 uM Reverse primer, SYBR-Green (0.12×), and fluorescein (10 nM), on ice. Tubes were transferred to a Bio-Rad “iCycler” thermal cycler fitted with an iQ real-time PCR detection module. Amplifications were run isothermally at 60° C. and monitored in real-time in the SYBR-Green channel for 1 hour. Each positive reaction was represented by a characteristic dye-binding curve for which a “Response Time,” analogous to “Ct” or cycle number for real time PCR assays, can be computed. The Rt (or Ct) is a standard measure of comparison of dye binding curves in real time amplification experiments, and is well understood to be inversely related to the log of the input number of targets (in PCR) or circular probes (in RAM) assays. That is, greater target (or circular probe) input results in lower (i.e., faster) Ct (or Rt) values respectively.

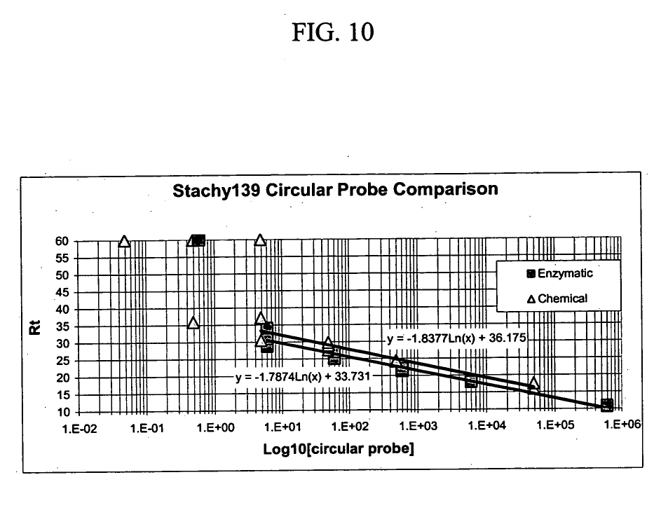

FIG. 10 demonstrates that the chemically and enzymatically synthesized circular probes replicate with virtually identical kinetics (i.e., the slopes are virtually identical), except that the dose response curves are offset by about 3.3 circular probes. That is, for example, 130 enzymatically produced circular probes result in Rt's of about 25 minutes whereas it takes about 438 chemically produced circular probes to generate a 25 minute response time. In general, having examined multiple manufacturing lots of multiple chemically synthesized circular probes we have observed wide variation in the apparent level of improved replication behavior. One consistent observation is that all enzymatically produced circular probes replicate consistently well, whereas there is wide variation in the replication properties of chemically synthesized probes, even within multiple lots of the same sequence from the same vendor. Examples of over 100-fold improvement have been observed, although about 10-fold improvement is most common. Occasionally, as in this example, a more modest, but still significant, levels of improvement are observed.

Various publications are cited herein, the contents of which are hereby incorporated by reference in their entireties.

We claim:

1. A method for producing circular probes comprising:
(a) contacting a first spanning oligonucleotide under conditions that allow nucleic acid hybridization between complementary sequences in the spanning oligonucleotide with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable probe, wherein the circularizable probe is bound on its 3′ and 5′ ends to the adjacent but not overlapping sequences in the spanning oligonucleotide;
(b) ligating the 3′ and 5′ ends of the circularizable probe with a ligating agent that joins nucleotide sequences such that a circular probe is formed;
(c) amplifying the circular probe of step (b) by contacting the circular probe of step (b) with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe;
(d) cleaving the single stranded DNA of repeating units with a restriction enzyme, under conditions whereby the restriction enzyme cleaves the single stranded DNA of repeating units into single unit length oligonucleotides;
(e) contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide of step (d);
(f) repeating steps (b) through (d);
(g) contacting the first spanning oligonucleotide under conditions that allow nucleic acid hybridization between complementary sequences in the first spanning oligonucleotide with at least one unit length oligonucleotide produced from step (f); and
(h) ligating the 3′ and 5′ ends of the unit length oligonucleotide produced from step (g) with a ligating agent that joins nucleotide sequences such that a circular probe is formed.

2. A circular probe made according to the method of claim 1.

3. The method of claim 1, wherein the DNA polymerase is φ29 DNA polymerase or Bst DNA polymerase.

4. The method of claim 1, wherein the ligating agent is an enzyme or a chemical agent.

5. The method of claim 4, wherein the enzyme is a DNA ligase.

6. The method of claim 5, wherein the DNA ligase is T₄ DNA ligase or Taq DNA ligase.

7. The method of claim 4, wherein the chemical agent is cyanogen bromide.

8. An amplification kit comprising:
(a) at least one first spanning oligonucleotide;
(b) at least one circularizable probe;
(c) at least one extension primer; and
(d) at least one second spanning oligonucleotide.

9. A method for producing linear circularizable probes comprising:
(a) contacting a first spanning oligonucleotide under conditions that allow nucleic acid hybridization between complementary sequences in the spanning oligonucleotide with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable probe, wherein the circularizable probe is bound on its 3′ and 5′ ends to the adjacent but not overlapping sequences in the spanning oligonucleotide;
(b) ligating the 3′ and 5′ ends of the circularizable probe with a ligating agent that joins nucleotide sequences such that a circular probe is formed;
(c) amplifying the circular probe of step (b) by contacting the circular probe of step (b) with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe;
(d) cleaving the single stranded DNA of repeating units with a restriction enzyme, under conditions whereby the restriction enzyme cleaves the single stranded DNA of repeating units into single unit length oligonucleotides;
(e) contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide of step (d); and
(f) repeating steps (b) through (d) such that linear circularizable probes are formed.

10. A linear circularizable probe made according to the method of claim 10.

11. The method of claim 9, wherein the DNA polymerase is φ29 DNA polymerase or Bst DNA polymerase.

12. The method of claim 9, wherein the ligating agent is an enzyme or a chemical agent.

13. The method of claim 12, wherein the enzyme is a DNA ligase.

14. The method of claim 13, wherein the DNA ligase is T₄ DNA ligase or Taq DNA ligase.

15. The method of claim 12, wherein the chemical agent is cyanogen bromide.

16. A method of detecting a target nucleic acid in a sample comprising:
(a) contacting a first spanning oligonucleotide under conditions that allow nucleic acid hybridization between complementary sequences in the spanning oligonucleotide with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable probe, wherein the circularizable probe is bound on its 3′ and 5′ ends to the adjacent but not overlapping sequences in the spanning oligonucleotide;
(b) ligating the 3′ and 5′ ends of the circularizable probe with a ligating agent that joins nucleotide sequences such that a circular probe is formed;
(c) amplifying the circular probe of step (b) by contacting the circular probe of step (b) with an extension primer that is complementary and hybridizable the circular probe, dNTPs, and a DNA polymerase having strand displacement activity, under conditions whereby the extension primer is extended around the circular probe for multiple revolutions to form a single stranded DNA of repeating units complementary to the sequence of the circular probe;
(d) cleaving the single stranded DNA of repeating units with a restriction enzyme, under conditions whereby the restriction enzyme cleaves the single stranded DNA of repeating units into single unit length oligonucleotides;
(e) contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide of step (d);
(f) repeating steps (b) through (d);
(g) contacting the first spanning oligonucleotide under conditions that allow nucleic acid hybridization between complementary sequences in the first spanning oligonucleotide with at least one unit length oligonucleotide produced from step (f);
(h) ligating the 3′ and 5′ ends of the unit length oligonucleotide produced from step (g) with a ligating agent that joins nucleotide sequences such that a circular probe is formed;
(i) contacting the target nucleic acid under conditions that allow nucleic acid hybridization between complementary sequences in the target nucleic acid with at least one circular probe produced from step (h), the circular probe comprising regions that are complementary to adjacent but not overlapping sequences in the target nucleic acid, the complementary regions separated by a generic region that is neither complementary nor hybridizable to a nucleotide sequence in the target nucleic acid, such that a complex is formed comprising the target nucleic acid and the circular probe;
(j) amplifying the circular probe of the complex of step (i) by contacting the circular probe of the complex of step (i) with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe; and
(k) detecting the single stranded DNA of repeating units, wherein detection thereof indicates the presence of the target nucleic acid in the sample.

17. The method of claim 16, wherein the DNA polymerase is φ29 DNA polymerase or Bst DNA polymerase.

18. The method of claim 16, wherein the ligating agent is an enzyme or a chemical agent.

19. The method of claim 18, wherein the enzyme is a DNA ligase.

20. The method of claim 19, wherein the DNA ligase is T₄ DNA ligase or Taq DNA ligase.

21. The method of claim 18, wherein the chemical agent is cyanogen bromide.

22. A method of detecting a target nucleic acid in a sample comprising:
(a) contacting a first spanning oligonucleotide under conditions that allow nucleic acid hybridization between complementary sequences in the spanning oligonucleotide with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable probe, wherein the circularizable probe is bound on its 3′ and 5′ ends to the adjacent but not overlapping sequences in the spanning oligonucleotide;
(b) ligating the 3′ and 5′ ends of the circularizable probe with a ligating agent that joins nucleotide sequences such that a circular probe is formed;
(c) amplifying the circular probe of step (b) by contacting the circular probe of step (b) with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe;
(d) cleaving the single stranded DNA of repeating units with a restriction enzyme, under conditions whereby the restriction enzyme cleaves the single stranded DNA of repeating units into single unit length oligonucleotides;
(e) contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide of step (d);
(f) repeating steps (b) through (d);
(g) contacting the first spanning oligonucleotide under conditions that allow nucleic acid hybridization between complementary sequences in the first spanning oligonucleotide with at least one unit length oligonucleotide produced from step (f);
(h) ligating the 3′ and 5′ ends of the unit length oligonucleotide produced from step (g) with a ligating agent that joins nucleotide sequences such that a circular probe is formed;
(i) contacting the target nucleic acid under conditions that allow nucleic acid hybridization between complementary sequences in the target nucleic acid with at least one circular probe produced from step (h), the circular probe comprising regions that are complementary to adjacent but not overlapping sequences in the target nucleic acid, the complementary regions separated by a generic region that is neither complementary nor hybridizable to a nucleotide sequence in the target nucleic acid, such that a complex is formed comprising the target nucleic acid and the circular probe;
(j) amplifying the circular probe of the complex of step (i) by contacting the circular probe of the complex of step (i) with a first extension primer that is complementary and hybridizable to the circular probe, a second extension primer that is substantially identical to portions of the circular probe, dNTPs, and a DNA polymerase having strand displacement activity, under conditions whereby the extension primer is extended around the circular probe for multiple revolutions to form a single stranded DNA of repeating units complementary to the sequence of the circular probe, and multiple copies of the second extension primer hybridize to complementary regions of the single stranded DNA and are extended by the DNA polymerase to provide extension products; and
(k) detecting the extension products, wherein detection thereof indicates the presence of the target nucleic acid in the sample.

23. The method of claim 22, wherein the DNA polymerase is φ29 DNA polymerase or Bst DNA polymerase.

24. The method of claim 22, wherein the ligating agent is an enzyme or a chemical agent.

25. The method of claim 24, wherein the enzyme is a DNA ligase.

26. The method of claim 25, wherein the DNA ligase is T₄ DNA ligase or Taq DNA ligase.

27. The method of claim 24, wherein the chemical agent is cyanogen bromide.

28. A method of detecting a target nucleic acid in a sample comprising:
(a) contacting a first spanning oligonucleotide under conditions that allow nucleic acid hybridization between complementary sequences in the spanning oligonucleotide with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable probe, wherein the circularizable probe is bound on its 3′ and 5′ ends to the adjacent but not overlapping sequences in the spanning oligonucleotide;
(b) ligating the 3′ and 5′ ends of the circularizable probe with a ligating agent that joins nucleotide sequences such that a circular probe is formed;
(c) amplifying the circular probe of step (b) by contacting the circular probe of step (b) with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe;
(d) cleaving the single stranded DNA of repeating units with a restriction enzyme, under conditions whereby the restriction enzyme cleaves the single stranded DNA of repeating units into single unit length oligonucleotides;
(e) contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide of step (d);
(f) repeating steps (b) through (d) such that linear circularizable probes are formed;
(g) contacting the target nucleic acid in the sample under conditions that allow nucleic acid hybridization between complementary sequences in the target nucleic acid with at least one circularizable probe produced in step (f) having 3′ and 5′ regions that are complementary to adjacent but not overlapping sequences in the target nucleic acid, the 3′ and 5′ regions separated by a generic region that is neither complementary nor hybridizable to a nucleotide sequence in the target nucleic acid, such that a complex is formed comprising the target nucleic acid and the circularizable probe, wherein the circularizable probe is bound on its 3′ and 5′ ends to the adjacent but not overlapping sequences in the target nucleic acid;
(h) ligating the 3′ and 5′ ends of the circularizable probe utilized in step (g) with a ligating agent that joins nucleotide sequences such that a circular probe is formed;
(i) amplifying the circular probe of step (h) by contacting the circular probe of step (h) with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe; and
(j) detecting the single stranded DNA of repeating units, wherein detection thereof indicates the presence of the target nucleic acid in the sample.

29. The method of claim 28, wherein the DNA polymerase is φ29 DNA polymerase or Bst DNA polymerase.

30. The method of claim 28, wherein the ligating agent is an enzyme or a chemical agent.

31. The method of claim 30, wherein the enzyme is a DNA ligase.

32. The method of claim 31, wherein the DNA ligase is T₄ DNA ligase or Taq DNA ligase.

33. The method of claim 30, wherein the chemical agent is cyanogen bromide.

34. A method of detecting a target nucleic acid in a sample comprising:
(a) contacting a first spanning oligonucleotide under conditions that allow nucleic acid hybridization between complementary sequences in the spanning oligonucleotide with at least one oligonucleotide probe, the oligonucleotide probe comprising a circularizable probe having 3′ and 5′ regions that are complementary to adjacent but not overlapping sequences in the spanning oligonucleotide, such that a complex is formed comprising the spanning oligonucleotide and the circularizable probe, wherein the circularizable probe is bound on its 3′ and 5′ ends to the adjacent but not the overlapping sequences in the spanning oligonucleotide;
(b) ligating the 3′ and 5′ ends of the circularizable probe with a ligating agent that joins nucleotide sequences such that a circular probe is formed;
(c) amplifying the circular probe of step (b) by contacting the circular probe of step (b) with an extension primer that is complementary and hybridizable to the circular probe, dNTPs, and a DNA polymerase having strand displacement activity, under conditions whereby the extension primer is extended around the circular probe for multiple revolutions to form a single stranded DNA of repeating units complementary to the sequence of the circular probe;
(d) cleaving the single stranded DNA of repeating units with a restriction enzyme, under conditions whereby the restriction enzyme cleaves the single stranded DNA of repeating units into single unit length oligonucleotides;
(e) contacting a second spanning oligonucleotide, comprising nucleic acid sequences complementary to the unit length oligonucleotide, under conditions that allow nucleic acid hybridization between complementary sequences in the second spanning oligonucleotide with at least one unit length oligonucleotide of step (d);
(f) repeating steps (b) through (d) such that linear circularizable probes are formed;
(g) contacting the target nucleic acid in the sample under conditions that allow nucleic acid hybridization between complementary sequences in the target nucleic acid with at least one circularizable probe produced in step (f) having 3′ and 5′ regions that are complementary to adjacent but not overlapping sequences in the target nucleic acid, the 3′ and 5′ regions separated by a generic region that is neither complementary nor hybridizable to a nucleotide sequence in the target nucleic acid, such that a complex is formed comprising the target nucleic acid and the circularizable probe, wherein the circularizable probe is bound on its 3′ and 5′ ends to the adjacent but not overlapping sequences in the target nucleic acid;
(h) ligating the 3′ and 5′ ends of the circularizable probe utilized in step (g) with a ligating agent that joins nucleotide sequences such that a circular probe is formed;
(i) amplifying the circular probe of step (h) by contacting the circular probe of step (h) with a first extension primer that is complementary and hybridizable to the circular probe, a second extension primer that is substantially identical to portions of the circular probe, dNTPs, and a DNA polymerase having strand displacement activity, under conditions whereby the extension primer is extended around the circular probe for multiple revolutions to form a single stranded DNA of repeating units complementary to the sequence of the circular probe, and multiple copies of the second extension primer hybridize to complementary regions of the single stranded DNA and are extended by the DNA polymerase to provide extension products; and
(j) detecting the extension products, wherein detection thereof indicates the presence of the target nucleic acid in the sample.

35. The method of claim 34, wherein the DNA polymerase is φ29 DNA polymerase or Bst DNA polymerase.

36. The method of claim 34, wherein the ligating agent is an enzyme or a chemical agent.

37. The method of claim 36, wherein the enzyme is a DNA ligase.

38. The method of claim 37, wherein the DNA ligase is T₄ DNA ligase or Taq DNA ligase.

39. The method of claim 36, wherein the chemical agent is cyanogen bromide.


SEQUENCE LISTING (6 sequences)

SEQ ID NO: 1 - DNA, 32 bp, Artificial
  artificial sequence assay probe
cactcagagaatactgaaaaaaacacaagagt

SEQ ID NO: 2 - DNA, 88 bp, Artificial
  artificial sequence assay probe
gcgcctttccagacgcttaccaagagcaactacacgaattctcgattaggttactgcgattagcacaagcgctgtcaccctgtatcgc

SEQ ID NO: 3 - DNA, 124 bp, Artificial
  artificial sequence assay probe
ggttttgtcttcgtaactcgctccggatgtctgtgtatctgctaaccaagagcaactacacgaattctcgattaggttactgcgattagcacaagctctacaagagtacatcggtcaacgaaga

SEQ ID NO: 4 - DNA, 110 bp, Artificial
  artificial sequence assay probe
agtattctctgagtggcaaacgcaatgaagcttgtcctagtgtgtcagtcgcacgcttaccaagagcaactacacgaacagctgtgaccccaaactcttgtgtttttttc

SEQ ID NO: 5 - DNA, 95 bp, Artificial
  artificial sequence assay probe
agtattctctgagtggcaaacgcaatgaagcttgtcctagtgtgtcagtcgcacgcttaccaagagcaactacacgaacagctgttgtttttttc

SEQ ID NO: 6 - DNA, 19 bp, Artificial
  artificial sequence assay probe
acacgaacagctgtgaccc